(19) United States
(12) Patent Application Publication
(10) Pub. No.: US 2006/0095058 A1
(43) Pub. Date: May 4, 2006

(54) Prostate treatment stent

(75) Inventors: Tovy Sivan, Kfar-Saba (IL); Ido Kilemnik, Herzelia (IL)

Correspondence Address: WOLF, BLOCK, SCHORR & SOLIS-COHEN LLP, 250 PARK AVENUE, NEW YORK, NY, 10177, US

(73) Assignee: Protech Medical Technologies Ltd., Bat-Yam (IL)

(21) Appl. No.: 11/248,733

(22) Filed: Oct. 12, 2005

(30) Foreign Application Priority Data
Oct. 13, 2004 (IL) 164563

(51) Int. Cl. A61B 17/32; A61B 18/18

(52) U.S. Cl. 606/170; 606/45; 606/174

(57) ABSTRACT

A method of dissecting urethra obstructing tissue. The method includes capturing urethra obstructing tissue between portions of an implant and applying pressure on the tissue caught between the portions of the implant, for longer than 1 hour, until the tissue dies or falls off.

### PRIORITY APPLICATIONS

The present application claims priority from Israel patent application 164563, filed on Oct. 13, 2004, the disclosure of which is incorporated herein by reference.

### FIELD OF THE INVENTION

The present invention relates to the field of prostate treatment.

### BACKGROUND OF THE INVENTION

Benign prostate enlargement is a common affliction among older men. Prostate enlargement involves swelling of the prostate, which blocks the urinary path. A common treatment for prostate enlargement is resection, which includes cutting away a portion of the prostate gland. The resection may be performed by a scalpel inserted through the penis to the prostate using a resectoscope. In such a procedure, however, the view of a physician performing the resection is limited and a mistaken move of the physician may cause serious damage to the patient. Other methods of treatment of benign prostate enlargement include heat treatment and/or low temperature ablation.

U.S. Pat. No. 5,928,217 to Mikus et al., the disclosure of which is incorporated herein by reference, describes a stent for placement in the urethra, for heating the prostate.

U.S. Pat. No. 5,588,965 to Burton et al., the disclosure of which is incorporated herein by reference, describes a device which gradually applies radial pressure against the prostate enlargement in order to slowly dilate the obstructed portion of the urethra. The device expands 5-20 French within 24 hours.

In other cases, a stent is implanted in the prostate to keep it open.

U.S. Pat. No. 5,601,591 to Edwards et al., the disclosure of which is incorporated herein by reference, describes a stent for introduction into the urethra. The stent is formed of coils spaced from each other, by between 1-2 millimeters, in order to allow urethra tissue to enter into spaces between the coils, for anchoring.

The long term implantation of a stent in the prostate, however, is problematic in itself. U.S. Pat. No. 6,416,545 to Mikus et al., the disclosure of which is incorporated herein by reference, describes a removable stent. The stent is used to heat the prostate in addition to supporting the prostate. After the prostate is substantially healed the stent is cooled and removed from the patient's body.

U.S. Pat. No. 6,238,368 to Devonec, the disclosure of which is incorporated herein by reference, describes a stent which both supports the prostate and provides a therapeutic agent which is cytoreductive to the prostate.

PCT publication WO 03/101311, the disclosure of which is incorporated herein by reference, describes a shape memory clip used to connect portions of an intestine. The clip presses portions of the intestine together. When the intestine sufficiently heals, a portion of the intestine held by the clip dies, such that the clip falls into the intestine and is evacuated from the patient's body.

U.S. Pat. No. 6,460,542 to James, the disclosure of which is incorporated herein by reference, mentions prosthetic devices for female bladder support. The James patent warns from the prosthetic devices exerting too much pressure, which may cause necrosis.

### SUMMARY OF THE INVENTION

An aspect of some embodiments of the invention relates to an implant device for removal of blocking tissue from the urethra. The device traps blocking tissue and applies force to the trapped tissue in order to cause the blocking tissue to fall out of the way and exit the urethra, widening the urethra. In some embodiments of the invention, the device reshapes the urethra to a desired open configuration. The force is optionally applied gradually over a long period, so as to avoid traumatic effects on the tissue.

The applied force is expected to starve or otherwise kill the cells of the tissue trapped by the device. In some embodiments of the invention, the force is applied for between about 7-30 days, although the device may be configured for shorter or longer time periods and/or the device may be used, in some cases for longer or shorter than the configured period. In an exemplary embodiment of the invention, a force of between about 100-300 grams is applied by the device. Optionally, the device applies a force of less than 250 grams, less than 150 grams or even less than 100 grams. In some embodiments of the invention, portions of the device move at a low rate of less than 1 mm in 24 hours, or even less than half a millimeter in 24 hours.

In some embodiments of the invention, the device is coated with a drug, for example, in order to aid the dissection process, counteract inflammation and/or counteract infection.

In some embodiments of the invention, the device includes a coil having a plurality of turns and the tissue is held between adjacent turns of the coil. In other embodiments, the device comprises a cylindrical tube with slits cut therein. In some embodiments of the invention, the device includes small protrusions (e.g., teeth) which penetrate the dissected tissue and enhance the dissection. Alternatively or additionally, at least some of the surfaces of the device which are intended to contact tissue are roughened, to an extent at which the device cannot be moved against tissue unless a substantial force is applied. Optionally, the roughened surfaces have a plurality of peaks having heights of at least 50 micrometers, 100 micrometers or even 300 micrometers.

In some embodiments of the invention, the rough surfaces are achieved by mechanical etching, for example rubbing sand paper or glass paper against the surfaces to be roughened and/or by laser etching. Alternatively or additionally, the rough surfaces are achieved by dipping the implant in an etching solution which roughens the surfaces of the implant, for example by random eating away of material of the implant. Further alternatively or additionally, the rough surfaces are achieved by a dipping the implant in a solution which causes irregular growth of a coating on the implant.

In some embodiments of the invention, the implant device is elastic, using any bio-compatible device structure and/or materials, such as super elastic materials, a bimetal structure and/or shape memory materials. Alternatively, a non-elastic device is used to apply the force, for example a device with a ratchet mechanism. Optionally, an external energy source is used to apply the force, for example a heat source and/or a magnet. Alternatively, an internal power source (e.g., battery) is located on the device within the patient. Alternatively or additionally, an external force may be applied mechanically by a handle of the device located outside the patient.

The device is constructed, in some embodiments of the invention, to contract radially in parallel to its axial contraction, to allow easier removal or even automatic expulsion from the urethra when its dissection task is completed.

In other embodiments of the invention, the implant expands radially with the axial contraction, so as to provide support against expanding tissue blocking the urethra. In still other embodiments of the invention, the implant is designed to have a complex radial behavior. For example, at first the implant optionally expands in order to increase pressure on the tissue and towards completion of the removal of the blocking tissue, the implant contracts radially, in order to allow easy removal thereof.

An aspect of some embodiments of the invention relates to a medical kit provided with an elastic tissue dissection device mounted in a stretched configuration on an insertion apparatus of the device. The dissection device and insertion apparatus are optionally provided in a sterile package.

An aspect of some embodiments of the invention relates to a method of inserting an elastic implant into the urethra. The method includes mounting the implant in a stretched state onto a delivery apparatus and inserting the implant into the urethra. Optionally, the stretched state comprises an axially stretched state. Alternatively or additionally, in the stretched state, the implant is wound around its axis a number of times, so as to decrease the radius of the elastic implant relative to a passive state of the implant.

Optionally, the elastic implant is held in the stretched state by the delivery apparatus. After the elastic implant is in position where it is to be released, the implant is released from the delivery apparatus.

When the implant is stretched axially, urethra obstructing tissue optionally enters gaps in the stretched elastic implant. The delivery apparatus is then caused to release the implant. Optionally, the elastic implant contracts axially, so as to trap the tissue that entered the gaps and apply elastic force at the trapped tissue.

Alternatively or additionally, the elastic implant is held open by sugar or any other dissolvable material. Inside the urethra, the sugar dissolves, obstructing tissue enters gaps in the elastic implant and the implant contracts so as to apply force on tissue trapped in the gaps.

In some embodiments of the invention, in which the implant is wound around itself, before the implant is released within the patient, the implant is rewound state in which the winding is cancelled.

An aspect of some embodiments of the invention relates to a two step method of safely treating prostate tissue without damaging the sphincter. The method includes inserting to the urethra, in a first stage, an overtube, which is adapted to allow determination of an extent of insertion of the overtube into the urethra, together with viewing apparatus for determining the location of the sphincter. Optionally, the viewing apparatus comprises an optical fibre, located within the overtube. The sphincter is located using the viewing apparatus and the extent of penetration of the overtube is recorded. Alternatively or additionally, the overtube is anchored in the patient, such that the distal end of the overtube protects the sphincter from damage. Thereafter, in a second stage, the viewing apparatus is removed from the outer tube, and a tissue treatment apparatus is passed through the outer tube to treat the tissue.

There is therefore provided in accordance with an exemplary embodiment of the invention, a method of dissecting urethra obstructing tissue, comprising capturing urethra obstructing tissue between portions of an implant and applying pressure on the tissue caught between the portions of the implant, for longer than 1 hour, 6 hours, or even 12 hours, optionally until the tissue dies or falls off.

Optionally, the implant applies different pressure levels along a length corresponding to an axis of the urethra. Alternatively, the implant applies substantially equal pressure along its length. Optionally, the implant comprises an elastic device. Optionally, applying the pressure comprises applying pressure for at least a day or even for at least a week.

Optionally, the implant comprises sharp tips pointed outward from the implant.

Optionally, the implant comprises tips pointed in directions in which the implant is adapted to capture tissue. Optionally, applying the pressure comprises applying pressure parallel to the axis of the urethra. Optionally, capturing tissue comprises inserting the implant into the urethra in a stretched state and releasing the implant such that it contracts and grasps tissue while contracting. Optionally, applying the pressure comprises applying by a non-elastic implant. Optionally, capturing tissue by an implant comprises inserting to the urethra an implant coated by a bio-active material. Optionally, the bio-active material comprises a tissue dissection drug. Optionally, the drug comprises a counter inflammation drug. Optionally, the implant comprises a coil. Optionally, capturing tissue comprises inserting the coil into the urethra in a winded state and rewinding the coil within the urethra. Optionally, the coil is formed of a wire having different thickness or cross-section shape along its length. Optionally, the coil has different length gaps between different pairs of turns of the coil. Optionally, the coil is pulled inside out in its production process. Optionally, the implant radially contracts when stretched axially.

There is further provided in accordance with an exemplary embodiment of the invention, a tissue dissecting implant kit, comprising an implant comprising a plurality of rings coupled to each other elastically, such that an elastic pressure is applied on tissue caught between adjacent rings and a sterile package encompassing the implant, wherein when the implant is in a stretched state resulting from pulling the implant from opposite ends, substantially the same pressure is applied between each pair of adjacent rings.

Optionally, the implant comprises a coil formed of a wire having different thickness or cross-section shape along its length.

There is further provided in accordance with an exemplary embodiment of the invention, a tissue dissecting implant kit, comprising an implant comprising a plurality of rings coupled to each other elastically, such that an elastic pressure is applied on tissue caught between adjacent rings and a sterile package encompassing the implant, wherein the implant contracts radially when it contracts axially, at a first axial stretching extent and expands radially when it contracts axially, at a second axial stretching extent.

Optionally, the implant comprises a coil.

There is further provided in accordance with an exemplary embodiment of the invention, a tissue dissecting implant kit, comprising an implant comprising a plurality of rings coupled to each other elastically, such that an elastic pressure is applied on tissue caught between adjacent rings and a sterile package encompassing the implant, wherein the implant has different distances between adjacent rings along its length or has different material thickness or cross-section shape along its length.

Optionally, the implant comprises a coil. Optionally, the implant has different distances between adjacent rings along its length.

There is further provided in accordance with an exemplary embodiment of the invention, a tissue dissecting implant kit, comprising an implant comprising a plurality of rings coupled to each other elastically, such that an elastic pressure is applied on tissue caught between adjacent rings and a sterile package encompassing the implant, wherein at least some of the ring surfaces facing each other have non-smooth surfaces.

Optionally, the non-smooth surfaces comprise rough surfaces having a feel similar to sand paper. Optionally, the non-smooth surfaces comprise small protrusions. Optionally, the rings are substantially circular or polygonal. Optionally, the implant comprises a coil. Alternatively, the implant comprises a cylinder with slits.

There is further provided in accordance with an exemplary embodiment of the invention, a tissue dissecting implant kit, comprising an elongate tube, sized and shaped to fit into a urethra, having a plurality of slits in a circumference of the tube, such that when the tube is stretched along its length it applies a contraction force on tissue within the slits and a sterile package in which the tube is packaged.

Optionally, the tube has a substantially cylinder shape in its rest state.

There is further provided in accordance with an exemplary embodiment of the invention, a tissue dissecting implant kit, comprising a sterile package and an axially elastic implant having a cylindrical shape, sized and shaped to fit in the urethra, the elastic implant adapted to apply a force of between about 100-1000 grams on tissue caught within the implant.

Optionally, the implant comprises pointed tips directed axially.

Optionally, at least a portion of the implant is coated with a material that counteracts inflammation. Optionally, at least a portion of the implant is coated with a material that counteracts tissue growth on the implant. Optionally, at least a portion of the implant is coated with a material that enhances tissue death. Optionally, the implant comprises a coil. Optionally, the implant comprises a cylindrical tube with slits cut therein. Optionally, the implant is adapted to contract radially when it contracts axially. Optionally, the implant is adapted to expand radially when it contracts axially.

There is further provided in accordance with an exemplary embodiment of the invention, a tissue dissecting implant, comprising an axially elastic implant adapted for insertion into the urethra and a bioactive material coupled to at least a portion of the implant.

Optionally, the bioactive material comprises a tissue dissection drug, a counter inflammation drug and/or a counter infection drug. Optionally, the bioactive material coats at least a portion of an outer surface of the implant. Optionally, the bioactive material coats at least some of the member surfaces facing another member. Optionally, the bioactive material coats at least a portion of the implant. Optionally, the bioactive material is embedded in at least a portion of the implant. Optionally, the implant comprises a plurality of members adapted to apply a dissecting force to tissue caught between the members.

There is further provided in accordance with an exemplary embodiment of the invention, a tissue dissecting implant, comprising an axially elastic implant adapted for insertion into the urethra, wherein the implant radially contracts when stretched axially.

There is further provided in accordance with an exemplary embodiment of the invention, a method of generating a medical implant for the urethra, comprising providing a coil suitable for implanting in the urethra and turning the coil inside out.

Optionally, providing the coil comprises providing a coil having at least ten turns.

Optionally, providing the coil comprises providing a coil having different distances between adjacent turns.

There is further provided in accordance with an exemplary embodiment of the invention, an implant delivery system for inserting an implant to the urethra, comprising a probe adapted to be inserted to a urethra, an implant holding unit adapted to hold an axially elastic implant in a stretched state on the probe and a release unit adapted to release the implant from the holding unit.

Optionally, the implant holding unit is adapted to hold the implant in an axially stretched state. Optionally, the implant holding unit is adapted to hold the implant in a winded state. Optionally, the release unit is adapted to rewind the implant. Optionally, the implant delivery system comprises a cover which separates the implant from the urethra until the implant is to be released. Optionally, the implant holding unit comprises strings that are torn by the release unit.

There is further provided in accordance with an exemplary embodiment of the invention, a method of implanting an implant into the urethra, comprising mounting the implant in a stretched state onto a probe suitable for insertion into the urethra, inserting the probe into the urethra, with the implant in the axially stretched state; and releasing the implant from the probe within the urethra, such that the implant axially condenses.

Optionally, mounting the implant in a stretched state comprises mounting in an axially stretched state. Optionally, the implant expands radially when it condenses axially. Optionally, the implant is released from the minimally invasive tool after tissue enters the implant in a manner which dampens the axial contraction of the implant. Optionally, mounting the implant in a stretched state comprises mounting in a wound state.

There is further provided in accordance with an exemplary embodiment of the invention, a method of treating the urethra, comprising inserting to the urethra an outer tube, inserting within the outer tube a viewing apparatus, setting a position of the outer tube in the urethra, which position protects the sphincter, using the viewing apparatus and applying a treatment to the urethra while the sphincter is protected by the outer tube.

Optionally, applying the treatment comprises removing the viewing apparatus from the outer tube and inserting a treatment probe through the outer tube. Optionally, applying the treatment comprises applying RF ablation, cutting tissue with a knife and/or implanting a tissue dissection implant. Optionally, the viewing apparatus comprises an optic fiber.

### BRIEF DESCRIPTION OF THE DRAWINGS

Particular exemplary embodiments of the invention will be described with reference to the following description of embodiments in conjunction with the figures, wherein identical structures, elements or parts which appear in more than one figure are generally labeled with a same or similar number in all the figures in which they appear, in which:

FIG. 1 is a schematic illustration of an elastic dissection implant, in accordance with an exemplary embodiment of the invention;

FIGS. 2A and 2B are schematic illustrations of stages in inserting an implant into a narrowed urethra path, in accordance with an exemplary embodiment of the invention;

FIGS. 3A and 3B are top and cross-sectional views of an implant insertion system, in accordance with an exemplary embodiment of the invention;

FIG. 3C and 3D are schematic illustrations of an implant delivery system, in accordance with an exemplary embodiment of the invention;

FIG. 4 is a flowchart of acts performed in inserting an implant into a patient, in accordance with an exemplary embodiment of the invention;

FIG. 5 is an enlarged view of a distal end of the insertion system of FIG. 3, in accordance with an exemplary embodiment of the invention;

FIGS. 6A and 6B schematically illustrate a process of removing an implant from a prostate, in accordance with an exemplary embodiment of the invention;

FIG. 6C schematically illustrates a process of removing an implant from a urethra, in accordance with another exemplary embodiment of the invention;

FIG. 7A is a schematic illustration of an elastic prostate implant, in accordance with an exemplary embodiment of the invention;

FIG. 7B is a schematic illustration of an elastic prostate implant, in accordance with another exemplary embodiment of the invention;

FIGS. 8A-8F are schematic illustrations of implant configurations, in accordance with an exemplary embodiment of the invention;

FIG. 9 is a schematic cross-sectional view of a tissue dissection tool, in accordance with an exemplary embodiment of the invention;

FIG. 10 is a schematic illustration of an implant insertion system, in accordance with an exemplary embodiment of the invention; and

FIGS. 11A-11E illustrate a method for inserting a reshaping implant, in accordance with an exemplary embodiment of the invention.

### DETAILED DESCRIPTION OF EXEMPLARY EMBODIMENTS

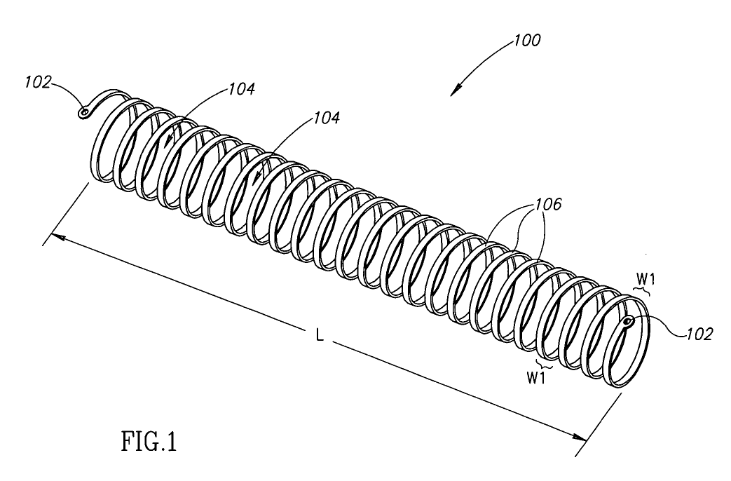

FIG. 1 is a schematic illustration of an elastic prostate implant 100, in accordance with an exemplary embodiment of the invention. Implant 100 includes an axially compressing device, such as a coil-shaped wire including a plurality of turns 106. Implant 100 has an elastic structure which axially compresses absent an external force. In FIG. 1, implant 100 is stretched such that gaps 104 are formed between turns 106. As described below, gaps 104 receive urethra-obstructing tissue which is to be removed and slowly press on the obstructing tissue until it is cut away and/or falls off due to tissue necrosis. In some embodiments of the invention, implant 100 includes anchoring structures, such as holes 102, at both ends, for use in stretching implant 100 to the state shown in FIG. 1. Holes 102 are optionally used during insertion of implant 100 to the urethra and/or for removal of the implant from the urethra.

Optionally, implant 100 is radially durable such that implant 100 does not collapse radially under pressure of the obstructing tissue. In some embodiments of the invention, while employed, implant 100 prevents blockage of urine passage, by allowing urine to pass through implant 100. Optionally, implant 100 also serves to expand urethra path 206 while the implant 100 is in the patient, by radially pushing the obstructing tissue.

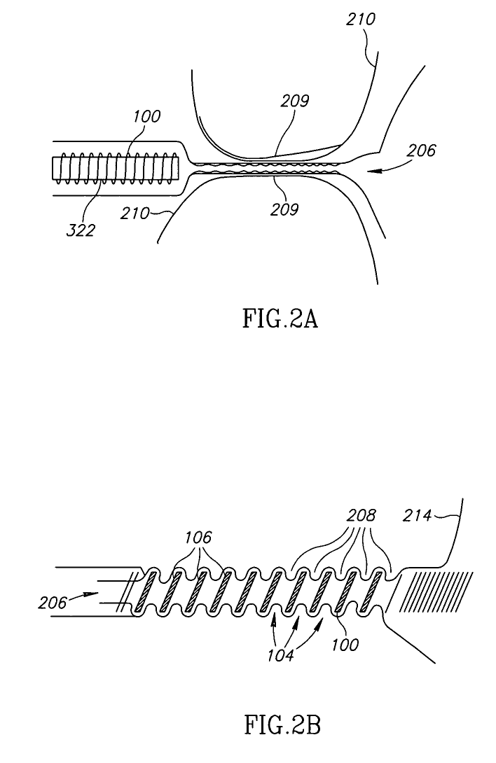

FIGS. 2A and 2B are schematic illustrations of stages in inserting implant 100 into a narrowed urethra path 206, in accordance with an exemplary embodiment of the invention. Urethra path 206 is narrowed by obstructing tissue 209 which is generally believed to block the urethra path due to a swelling of the prostate 210. Implant 100, optionally covered by a protective cover 322, is pushed into the urethra path 206, in an axially stretched orientation. Protective cover 322 is then removed, so as to allow portions 208 of obstructing tissue 209 to enter gaps 104 of implant 100, as shown in FIG. 2B.

Size

In an exemplary embodiment of the invention, the radius of implant 100 is large enough such that tissue portions 208 enter into gaps 104 between the turns 106 of the implant. In some embodiments of the invention, implant 100 has a radius of substantially the same size as of a stent known in the art, used to support the prostate and keep the urethra open. Alternatively, implant 100 has a slightly larger radius than a stent suggested to be employed on the patient, in order to allow more tissue to enter into gaps 104.

In some embodiments of the invention, physicians have a plurality of implants 100 of different sizes and a suitable implant is selected according to the patient. The patient's size may be determined from previous medical data and/or using imaging apparatus (e.g., of the obstruction, of the extent of the prostate growth) or insertion of a measurement device. In an exemplary embodiment of the invention, implant 100 has a radius of between about 3-11 millimeters, optionally between 4-10 millimeters. The gaps 104 between turns 106 are optionally sufficiently axially long to allow entrance of large amounts of tissue into the gaps, for example at least 0.5 millimeters, at least 2 mm or even at least 5 millimeters. Larger gaps (e.g., 7-11 mm, or even larger) are optionally used when the obstructing tissue is relatively hard or dense and/or when it is required to catch substantial amounts of tissue. In some embodiments of the invention, however, in order to allow for a substantial number of turns along implant 100, each turn has a length of less than 8 mm, less than 5 mm or even less than 2 mm. In an exemplary embodiment of the invention, smaller gaps 104, for example of 0.3-2 mm, are used. The size of gaps 104 optionally depends on the material forming implant 100. When a metal such as aluminum or stainless steel is used, gaps of up to about 5 mm are achievable. For Nitinol, gaps of even 15 mm are achievable.

In some embodiments of the invention, implant 100 has a length L slightly larger (e.g., 5-10% larger) than an obstruction treated, so as to treat the entire length of the obstruction at once. In some embodiments of the invention, implants 100 are provided in packages including implants of various sizes. Alternatively, the length L of implant 100 is as long as, or even longer than, any expected urethra obstruction to be treated. For smaller urethra obstructions, only part of implant 100 is operative, while the remaining part contracts within the patient's bladder 214 or within non-obstructed portions of the urethra, immediately after installation. Alternatively, a physician may shorten implant 100 by cutting one end of the implant, before installation in the patient. Alternatively or additionally, an implant 100 shorter than the obstruction is used. In accordance with this alternative, as well as the other alternatives, a plurality of consecutive treatment sessions may be performed. Optionally, in a first treatment session, an implant 100 with a relatively small radius is used. In following treatment sessions, implants with larger radius are used. Alternatively, a plurality of consecutive treatment sessions are performed with implants 100 of the same length.

In an exemplary embodiment of the invention, implant 100 has a length, when stretched, of between at least 50 millimeters, 60 millimeters or even at least 70 millimeters, according to the length of the urethra obstruction. The non-stretched length of implant 100 optionally depends on the material from which implant 100 is comprised. For stainless steel, a non-stretched length of about 40 mm corresponds to a stretched length of 60-70 millimeters, while for nitinol a length of 8-10 mm optionally corresponds to 60-70 millimeters.

In an exemplary embodiment of the invention, implant 100 includes between 15-35 turns 106 per centimeter in its rest state. Alternatively, a larger number of turns (e.g., 35-60) are included in each centimeter.

The tissue portions 208 entering into gaps 104 optionally anchor implant 100 within urethra path 206 so as to prevent implant 100 from being pushed out of the urethra path 206, for example by urinating. Alternatively or additionally, implant 100 includes radial protrusions which anchor the implant in place.

In some embodiments of the invention, the radius of implant 100 is substantially the same in a stretched state as in a contracted state. Alternatively, implant 100 is planned to expand radially when it contracts axially. In some embodiments of the invention, implant 100 is inserted into the patient axially stretched and radially contracted. When implant 100 is properly positioned in the patient, the axial stretching is released, so that implant 100 catches obstructing tissue in gaps 104. The radial expansion of implant 100 improves the anchoring of the implant in the patient's tissue and/or increases the amount of enlargement tissue caught by implant 100. In addition, the radial expansion with axial contraction allows easier insertion and/or removal of implant 100 in its axially stretched state. In other embodiments of the invention, implant 100 contracts radially when it contracts axially, so that it easily exits when the killing of the obstruction tissue is completed. In still other embodiments of the invention, in the maximally stretched state, implant 100 has a medium radius. In axially contracting from the maximally stretched state, the radius expands to a maximal radius. Thereafter, in continuous axial contraction, the radius reduces in order to allow easy removal of the implant.

Pressure

Implant 100 optionally applies a generally axial compressive pressure against tissue entering into gaps 104. The pressure applied by implant 100 is optionally relatively weak so as not to cause substantial pain to the patient. Alternatively or additionally, the pressure of implant 100 is set to a level that causes all the tissue entering into gaps 104 to cut off from the prostate within a predetermined time range, optionally between 1-4 weeks. The pressure, however, is not set to too low a level which is not sufficient for killing the tissue. In some embodiments of the invention, the pressure is large enough to cut off callus tissue which may obstruct the urethra. Alternatively, a low pressure implant 100 is used generally on patients, unless it is determined that the patient has (or may have) callus tissue, in which case a higher pressure implant is used.

In some embodiments of the invention, the pressure is set to gradually cut off the blood flow to the cells of the tissue enlargement, so that sudden ischemia which could cause gangrene does not occur. Optionally, experimentation is performed in order to determine a best pressure level. In some embodiments of the invention, different implants 100, which apply different pressure levels, are used on different patients according to one or more attributes of the patients, such as age, tissue hardness and/or existing inflammation.

In an exemplary embodiment of the invention, a starting force of at least 100 grams, for example between 150-300 grams is applied by implant 100 on the tissue within gaps 104. Alternatively, lower starting forces such as less than 100 grams (e.g., 30 grams) or even as low as 1-20 grams are used. Further alternatively, higher starting forces of at least 300 grams, 500 grams or even at least 800 grams are used, for example when the patient has very soft obstructing tissue or when the patient has very tough tissue.

The force applied by implant 100 optionally reduces with axial compression of the implant. In some embodiments of the invention, implant 100 includes a slowly degrading coating or other layer that reduces the force applied by implant 100. The degrading coating includes, for example, a sugar or a polymer that dissolves in water. The coating degrades with time, so that the force increases with time, in parallel to the decreasing of the force due to the axial contraction of implant 100. In some embodiments of the invention, the degrading coating is applied such that the force of implant 100 remains substantially constant over the entire treatment until implant 100 totally axially contracts. Alternatively, the coating is chosen to achieve a desired predetermined profile. The predetermined profile optionally has an increasing force profile, so as to take advantage of the patient's getting used to the applied force. Alternatively, a decreasing force profile is used. Further alternatively or additionally, a force profile which includes both increasing and decreasing segments is used.

In some embodiments of the invention, implant 100 is coated with a soft spongy coating in order to reduce the pressure applied by the implant when it is toward the end of its compression. These embodiments are optionally used when it is expected that the tissue toward the end of its dissection cannot withhold the force applied by the implant in its close to compressed state.

Implant 100 optionally applies different force along its length, for example stronger force being applied toward the axial center of the implant, as is the case in some simple springs. Alternatively, implant 100 is produced such that it applies the same force over its entire axial length. Further alternatively or additionally, implant 100 has any other axial force application profile. For example, an implant 100 to be used for a specific patient may be selected according to the axial distribution of the obstruction tissue and/or its hardness, in the patient.

The pressure profile of implant 100 is optionally controlled by changing the thickness and/or axial width of the material of implant 100 over its length. Alternatively or additionally, implant 100 is produced with different distances between adjacent turns along the length of the implant. Turns that are closer to each other in a rest state, optionally apply a stronger force than turns that are farther away in the rest state. If, however, as discussed below, implant 100 is pulled inside out in its production, turns that are farther from each other in their rest state apply a stronger force than closer turns.

In some embodiments of the invention, different axial portions of implant 100 are stretched to different lengths in order to control the pressure applied by the different portions. Optionally, implant 100 is designed to be stretched to different extents in different portions along its length. Generally, portions that are stretched to a greater extent apply more force on the tissue.

Alternatively or additionally, implant 100 has different thickness and/or axial width along its length, in order to achieve a desired pressure profile, which varies along the length of the implant.

The use of different pressure levels along the length of implant 100 optionally causes dissected tissue to fall off at different times. Causing dissected to fall off at different times, optionally reduces the chances of clogging the urethra with dissected tissue. In an exemplary embodiment of the invention, the pressure varies gradually from a high pressure on one end of implant 100 to a lower pressure on an opposite end of the implant. The high pressure end is optionally located on the end of the urethra closer to the body orifice, such that dissected tissue falling off earlier due to the high pressure is not clogged by the obstructing tissue not yet dissected, on its way out of the patient. Alternatively to gradual changes in the pressure, the pressure changes in steps.

In some embodiments of the invention, the pressure between each two turns 106 is directed at having the two turns adjacent each other, such that when the turns touch each other the pressure applied is substantially zero. Alternatively, the pressure between adjacent turns 106 is directed at having the turns change places, such that even when the turns 106 touch each other the turns apply pressure. In some embodiments of the invention, a spring which applies pressure even when the turns are adjacent each other is achieved by pulling a spiral implant inside out. Optionally, after producing the spiral, one end of the spiral is held stationary, while the other end of the spiral is pulled through the spiral, so as to turn it inside out. Optional materials to be used with a spiral implant pulled inside out are plastic, nitonol and/or nirosta.

In some embodiments of the invention, implant 100 is planned to advance in a certain direction, as it contracts axially. Optionally, implant 100 is formed of shape memory materials which induce the movement along with the axial contraction.

Operation

The slow operation of implant 100 prevents damage to the control valve (i.e., the sphincter) of the urethra due to a mistake of a physician. For example, if implant 100 is released on the sphincter, due to an inaccurate move on the part of a physician, the improper positioning of the implant can be corrected, even if the mistake is determined hours after the implant is employed.

Tissue cut off by the pressure of implant 100 generally falls into the path 204 and is optionally naturally washed out of the patient in the patient's urine stream. In some embodiments of the invention, the urine stream aids in detaching tissue, which is at least partially dissected. Alternatively, after the tissue is killed, the tissue remains connected to the urethra. A dissecting tool guided by implant 100 is used to remove the dead tissue. Further alternatively or additionally, the dead tissue separates from the urethra but remains in the urethra channel. An invasive tool is used to remove the dead tissue from the urethra path.

After all the tissue entering into gaps 104 is cut, implant 100 is optionally automatically freed from its anchoring to the urethra and is automatically expelled from the patient with the urine passing in path 204. Alternatively, implant 100 is actively removed, for example, using a medical procedure as described below. Further alternatively, after completing its task, implant 100 is dissolved or broken into pieces, which are removed with the urine, as described below. As described above, different implants 100 may be designed with different ending radius, such that a physician may select whether to use a self ejecting implant 100 or an implant which needs to be removed in a medical procedure.

Exemplary Materials of Implant

Implant 100 optionally comprises an elastic bio-compatible material, suitable for implantation in the urethra for a period of a few weeks. In some embodiments of the invention, implant 100 comprises a material (e.g., gold) which minimizes or totally inhibits tissue growth, bacterial growth and/or crystallization on the implant. Such tissue growth may interfere in the dissection operation of implant 100 and/or may limit the effectiveness of implant 100 in keeping the urine path of the patient open. Alternatively or additionally, implant 100 is coated with a suitable coating (e.g., gold) which prevents tissue growth.

Alternatively or additionally, implant 100 comprises a durable but cheap material, such as stainless steel and/or various plastics. In other embodiments of the invention, implant 100 comprises a super elastic material (e.g., nitinol, B-metal), allowing minimization of the material content of implant 100. The use of minimal material is sometimes desired when apparatus is used within a patient. In some embodiments of the invention, a shape memory material and/or a bi-metal structure is used for implant 100. The properties of the material may be used to externally control the force applied by implant 100 and/or to aid in insertion or removal of the implant. For example, the implant may be brought to a shrunken state for insertion or removal. In some embodiments of the invention, the external control is performed by heating and/or cooling the implant. Alternatively or additionally, the external control is performed by apply magnetic forces to the implant.

In some embodiments of the invention, implant 100 comprises a non-biodegradable material, such as any of the above mentioned materials. Alternatively, implant 100 comprises a bio-degradable material, which slowly dissolves within the urethra.

The bio-degradable materials used for implant 100 are optionally ones that degrade slowly, such that the implant falls apart only after the implant contracts substantially entirely. Optionally, materials that degrade near inert tissue and/or in urine, are used. Possible biodegradable materials for forming implant 100 are described, for example, in “Biodegradable, Elastic Shape Memory Polymers for Potential Biomedical Applications”, by Andreas Lendlein, Science Express, April 2002, the disclosure of which is incorporated herein by reference.

In some embodiments of the invention, implant 100 is entirely formed of a bio-degradable material (or a plurality of biodegradable), such that there is no need to remove implant 100 from the patient and/or it is easier to remove the implant from the patient. Alternatively, implant 100 is formed of bio-degradable and non-bio-degradable materials in interleaved sections, such that when the bio-degradable materials degrade, the non-bio-degradable materials are small enough to exit the body naturally and/or it is easier to remove them from the patient.

Optionally, implant 100 has a degradable structure such that the degradation of the implant becomes of structural meaning only after a predetermined time in which the urethra obstruction cells are expected to be entirely dissected. In some embodiments of the invention, implant 100 is formed such that once the degradation causes the structure to deteriorate, the total collapse and subsequent evacuation through the urine is fast (e.g., within a few hours or days). For example, implant 100 may be made of a relatively thick material which dissolves in layers. Only when the last layer of the implant begins to dissolve does the implant collapse and stop its dissection operation.

In an exemplary embodiment of the invention, implant 100 includes a degradable plastic structure on a nitinol string. After the plastic degrades, only the nitinol string remains, possibly with some plastic remnants thereon, and the nitinol string is removed from the patient.

Smoothness or Roughness

Implant 100 optionally has a smooth outer radial surface so as to minimize the interaction between the implant and outer prostate tissue not caught in gaps 104. The smoothness optionally prevents anchoring of implant 100. In some cases, the smoothness hampers the pressure of the elasticity and/or minimizes inflammation and/or pain. Alternatively, the outer radial surface is sandblasted and/or teethed in order to enhance cell death also radially and/or to prevent too strong a pressure being applied by the elasticity. In some embodiments of the invention, the elasticity of implant 100 is defined according to the expected radial friction with the prostate walls. In some embodiments of the invention in which friction is desired, a friction coating is used to provide the friction, for example a nano-particle coating. When a bioactive coating is used, the use of a nano-particle coating also increases the contact area between the drug an the tissue to interact with the drug.

In some embodiments of the invention, implant 100 includes axially directed teeth and/or sandblasting directed at gaps 104, which optionally enhance the cell killing of the implant, on tissue within gaps 104.

Coating

Implant 100 is optionally coated with a suitable drug, solution or other bioactive material which prevents growth of crystals and/or tissue on the implant. In some embodiments of the invention, the coating is passive, i.e., the coating includes a material (or materials) to which growth does not cling and/or which does not induce growth. Alternatively or additionally, the coating is active, i.e., the coating includes chemicals that attack growth and/or prevent its formation.

Alternatively or additionally to preventing growth, implant 100 is coated with a bioactive material which hastens decay of surrounding live and/or dead tissue. In some embodiments of the invention, the coating is placed on the entire surface area of the implant. Alternatively, only portions of the surface area of the implant are coated, for example inner surface areas and/or side portions facing gaps 104.

Further alternatively or additionally, implant 100 is coated with a fluorescent or other imaging aiding material, to aid in identifying the implant in medical images. In some embodiments of the invention, coating is used for other reasons, such as enlarging or decreasing the friction between the implant and the prostate.

Alternatively to being coated with the bioactive material, the bioactive material is embedded within the implant or is placed in a miniature pocket on or coupled to the implant. In some embodiments of the invention, the bioactive material is slowly released to the surrounding tissue over more than 24 hours or even over more than a week. Optionally, the bioactive material is released according to a predetermined scheme. Alternatively, the release of the bioactive material is induced by tissue inflammation, urinating or any other internal biological effect within the patient. Alternatively, the release of the bioactive material is induced externally from outside the patient, using any method known in the art. Further alternatively, the bioactive material may be coupled to the implant in any other method known in the art.

Structure

In some embodiments of the invention, implant 100 is formed of a wire which is turned into a spiral shape. Optionally, the wire has a circular or elliptical cross section. Alternatively, the wire has a square, rectangular, triangular, star or diamond cross-sectional shape, which applies more pressure on the tissue captured in gaps 104. In some embodiments of the invention, the points of the diamond and/or triangle are directed axially at the captured tissue. Alternatively or additionally, the points are directed radially inward toward tissue captured within the implant. Alternatively, implant 100 is smooth inward in order not to interfere passage through the implant. In some embodiments of the invention, the wire forming implant 100 comprises a flat (thin) rectangular shape.

The use of simple geometrical shapes allows relatively cheap production. In some embodiments of the invention, however, more complex wire shapes are used, for example, in order to include more points and/or flat areas in desired directions.

Alternatively to forming implant 100 from a wire, implant 100 is cut out of a tube or a flat sheet.

The width w1 (FIG. 1) of turns 106, is optionally substantially equal to the length of gaps 104 in the stretched state. Alternatively, the width w1 of turns 106 is smaller than gaps 104, with a ratio of between about 1:2 and 1:4. Alternatively, width w1 of turns 106 is smaller than gaps 104 in the stretched state. The ratio between the width of turns 106 and the length of gaps 104 affects the extent of stretching of the tissue walls of the urethra In some embodiments of the invention, the force applied by implant 100 is adjusted according to the expected stretching, in order not to cause too much stretching and/or undesired slippage of implant 100 within the urethra. In some embodiments of the invention, the width w1 of turns 106 is equal over the entire length of implant 100. Alternatively, the width w1 of the turns varies over the length of implant 100, for example in order to vary the pressure applied by implant 100 over its length.

In some embodiments of the invention, the shape of implant 100 is adjusted to limit the stretching and/or scarring of tissue. The shape of the implant is optionally adjusted, in some embodiments of the invention, in order to direct tissue scarring in a desired direction or location, which is, for example, better suitable for healing.

Insertion Method

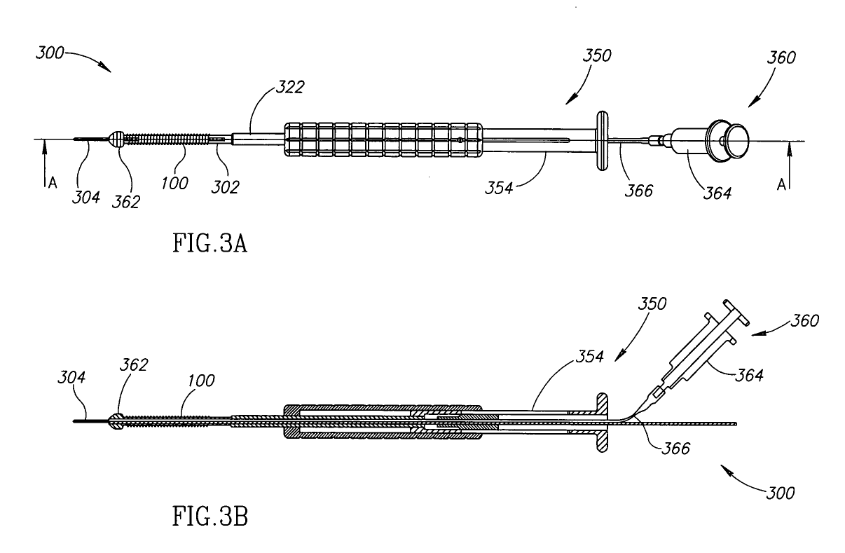

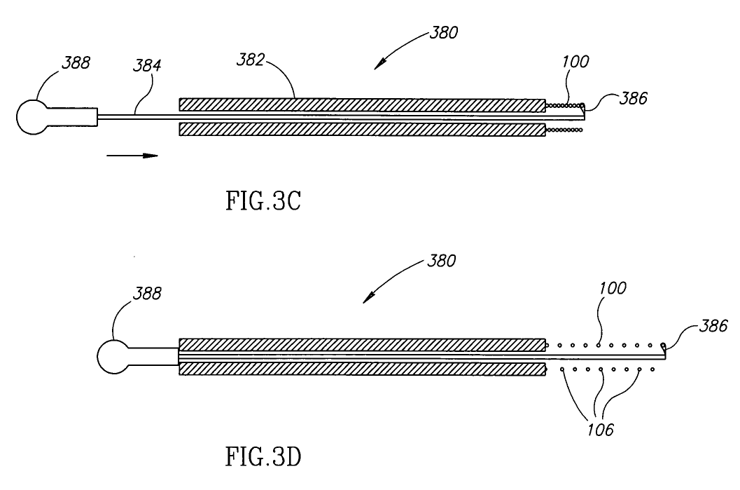

FIG. 3A is a top view of a probe system 300 for inserting implant 100 into the urethra of a patient, in accordance with an exemplary embodiment of the invention.

FIG. 3B is a cross-sectional view of system 300, in accordance with an exemplary embodiment of the invention. System 300 includes an implant carrier 350, which holds implant 100 in a stretched state.

In addition, system 300 optionally includes a balloon catheter 360, which aids in inserting and/or positioning implant 100 into the prostate. A balloon 362 at the distal end of balloon catheter 360 leads the way of implant 100 on its way along the urethra, opening the path of the urethra (which is blocked by obstructing tissue) and preventing damage to the implant. Alternatively, any other gadget is used to lead the way of implant 100, such as an umbrella or a protective cap. Implant carrier 350 optionally includes a bar 302 on which implant 100 is placed. An optional protective cover 322 protects implant 100 radially and/or protects the urethra from implant 100, while the implant is inserted into the patient. Balloon catheter 360 optionally includes, at its proximal end, a balloon control 364 which is used to inflate and/or deflate balloon 362. An inflation tube 366, passing through implant carrier 350, connects balloon 362 to balloon control 364.

Balloon catheter 360 and implant carrier 350 are optionally inserted to the patient on a guide wire 304. Alternatively or additionally, implant carrier 350 is inserted through a previously inserted tube and/or a catheter used for diagnosis.

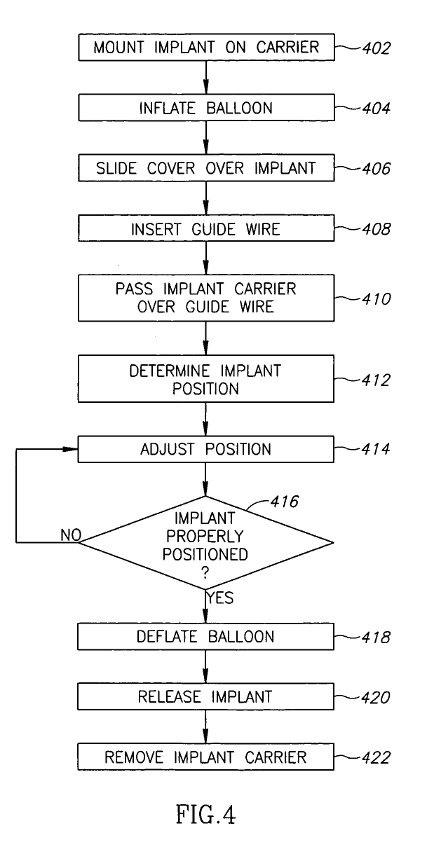

FIG. 4 is a flowchart of acts performed in inserting implant 100 to a patient, in accordance with an exemplary embodiment of the invention. Implant 100 is mounted (402) on implant carrier 350. Balloon 362 is inflated (404) and cover 322 is slid (406) over implant 100. A guide wire 304 is inserted (408) into the patient and implant carrier 350 and balloon catheter 360 are passed (410) into the patient together over guide wire 304. The position of implant 100 is determined (412) and the position is adjusted (414), until (416) the implant is properly positioned. Balloon 362 is then deflated (418) and implant carrier 350 releases (420) implant 100. Implant carrier 350, balloon catheter 360 and guide wire 304 are removed (422) from the patient.

Referring in more detail to mounting (402) implant 100 on carrier 350, in some embodiments of the invention, implant 100 is mounted on carrier 350 by the physician, immediately before insertion to the patient. In some embodiments of the invention, the physician cuts implant 100 into size before it is mounted. Alternatively, implant 100 is supplied pre-mounted on carrier 350. For example, in accordance with this alternative, carrier 350 may be supplied within a sterile package with implant 100 mounted thereon in a stretched state. In some embodiments of the invention, implant 100 is mounted on carrier 350 already stretched. Alternatively, implant 100 is set to a deformed state (e.g., using a shape memory material) which remains for at least a predetermined time required for implant insertion, but later allows the implant to retain to its usual pressure enforcing state.

FIG. 3C and 3D are schematic illustrations of an implant delivery system 380 suitable for stretching implant 100 immediately before insertion and/or after insertion, in accordance with an exemplary embodiment of the invention. As shown in FIG. 3C, delivery system 380 comprises a tubular body 382 and an internal shaft 384. Implant 100 is mounted on the distal end of tubular body 382, using any method known in the art, including methods described herein above. It is noted that, for simplicity, not all elements of the deliver system are shown in FIGS. 3C and 3D, for example a protective cover (corresponding to cover 322) may be included. It is noted, however, that if, for example, the stretching of implant 100 is performed within the urethra, a protective cover is not required and in some embodiments of the invention, is not deployed.

When implant 100 is to be stretched, a handle 388 of shaft 384 is pushed axially, so as to push a notch 386 coupled to implant 100 distally, and thus stretch implant 100, to the state shown in FIG. 3D.

Further alternatively or additionally, a stopper (not shown) is inserted into implant 100 holding it in its open state. When implant 100 is inserted into place the stopper is removed to allow the implant 100 to axially collapse and apply pressure on the obstruction tissue. In an exemplary embodiment of the invention, the stopper is in the form of a comb with prongs extending perpendicular to shaft 384, being placed in the gaps between the turns 106 of implant 100. Optionally, the stopper is elastic so that it can be removed by pulling handle 388 proximally. Alternatively or additionally, implant 100 has an elliptical cross-section. The stopper extends into implant 100 when it runs along the shorter axis of the implant. When it is to be removed, the stopper is rotated, by rotating handle 388, and then the stopper is removed.

In still other embodiments of the invention, implant 100 is stretched by a balloon placed within implant 100. When it is required to stretch the implant, the balloon is inflated. The shape of the balloon determines the extent to which tissue enters into gaps 104. After the tissue enters into the gaps of implant 100, the balloon is deflated and removed from the patient.

In some embodiments of the invention, before mounting implant 100 or before selecting a pre-mounted implant, the patient is diagnosed, in order to select an implant suitable for the patient. The diagnosis optionally includes imaging the obstructing tissue and/or inserting a measurement catheter for determining the patient's size and/or inflammation state.

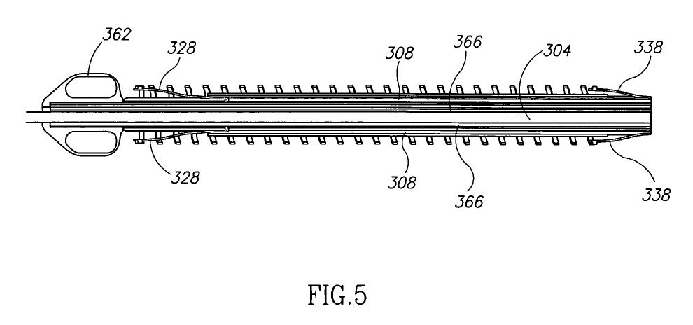

FIG. 5 is an enlarged view of a distal end of system 300, in accordance with an exemplary embodiment of the invention. Implant 100 is held by a sleeve 308 which surrounds inflation tube 366 and guide wire 304. Sleeve 308 carries levers 328 and 338 which are controllable from a handle 354 (FIG. 3A) of implant carrier 350. In a first state, levers 328 and 338 hold implant 100 in an expanded state while the implant is inserted into the patient. When implant 100 is determined to be properly positioned near the enlarged prostate tissue 108, levers 328 and 338 are moved into a release state, disconnecting implant 100 from implant carrier 350.

In some embodiments of the invention, sleeve 308 carries two distal levers 328 and two proximal levers 338. The use of two proximal levers 338 and two distal levers 328 provide stable holding of implant 100. Alternatively, more than four levers 328 and 338 may be used, or fewer than four levers may be used.

In some embodiments of the invention, the releasing (420) of implant 100 is performed simultaneously by all of levers 338 and 328. Alternatively, a slow release is performed, in order not to exert a large force on the tissue at the time of release. Optionally, distal levers 328 are released first and then proximal levers 338 are released, such that the proximal side of implant 100 remains in its position in the beginning of the obstructing tissue. Alternatively, the proximal levers 338 are released first. Further alternatively, each lever is released separately.

Optionally, before releasing implant 100, implant 100 is held in its stretched state for a sufficient time to allow obstructing tissue to enter gaps 104. Alternatively, implant 100 is pushed into place, such that obstructing tissue enters into the gaps 104 of the implant immediately upon insertion.

In some embodiments of the invention, an internal tube is positioned within implant 100 during the release of the implant, in order to limit the depth of entrance of tissue into the gaps. Optionally, the physician can select which of a plurality of internal tubes to use and/or whether to use a tube at all. Alternatively, the internal tube has portions of different radius. The physician controls the depth of entrance of obstructing tissue into gaps 104 by moving the internal tube axially.

Referring in more detail to determining (412) the position of implant 100, in some embodiments of the invention, the position is determined using an external imaging apparatus, such as x-ray or ultrasound. Alternatively or additionally, system 300 carries a viewing optical channel, for example within sleeve 308, which provides images of the urethra from inside. Further alternatively or additionally, system 300 is inserted until the additional resistance of enlargement area 202 against balloon 362 is felt by a physician performing the insertion. System 300 is then inserted an additional precise distance in order to bring implant 100 to enlargement area 202.

Alternatively or additionally, implant 100 or system 300 includes an expandable element (e.g., balloon 362) at its distal end. System 300 is pushed all the way into the patient's bladder 214, allowing the expandable element to expand and prevent pulling implant 100 away from the bladder 214. Thus, the physician knows that if balloon 362 was sufficiently expanded, a first end of implant 100 is positioned in the bladder 214 and the other end could not reach the patient's sphincter. Optionally, after implant 100 is properly positioned, the expandable element is collapsed and removed from the patient with system 300.

In some embodiments of the invention, implant 100 is left in place for a predetermined time, after which implant 100 is removed from the patient. Alternatively or additionally, images (e.g., ultrasound images) of implant 100 are acquired periodically (e.g., once every 3-9 days), to verify that no problems have occurred and/or to determine when the implant is to be removed from the patient. In the acquired images, the distance between neighboring turns 106 is optionally determined and accordingly the time until implant 100 completely contracts axially and needs to be removed, is determined. Optionally, the patient acquires the images on his own, and transmits the images to a physician for analysis.

While implant 100 is in place, its presence may be used to aid in surgical treatment procedures. In some embodiments of the invention, a tissue cutting apparatus is used to cut the tissue bulging into the center of the implant 100. The tissue cutting apparatus is optionally linked to the implant 100, which is distanced from the sphincter according to previous position verification, so that the tissue cutting apparatus does not inadvertently damage the sphincter.

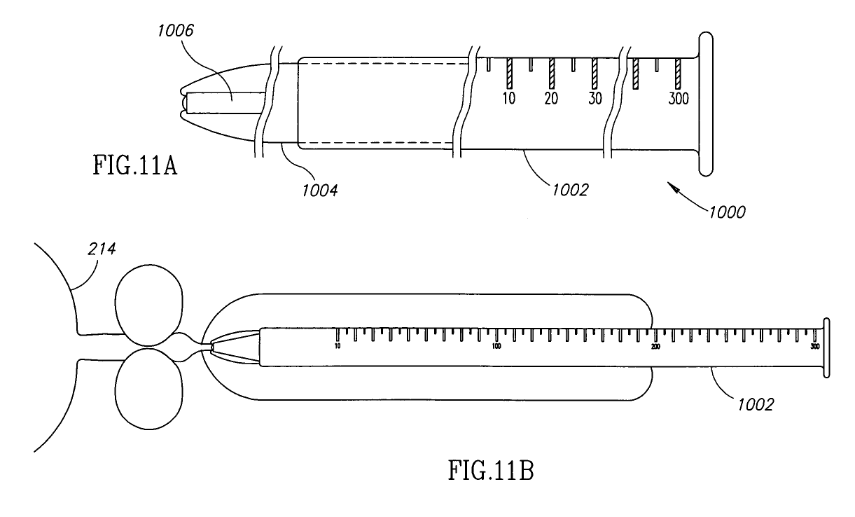

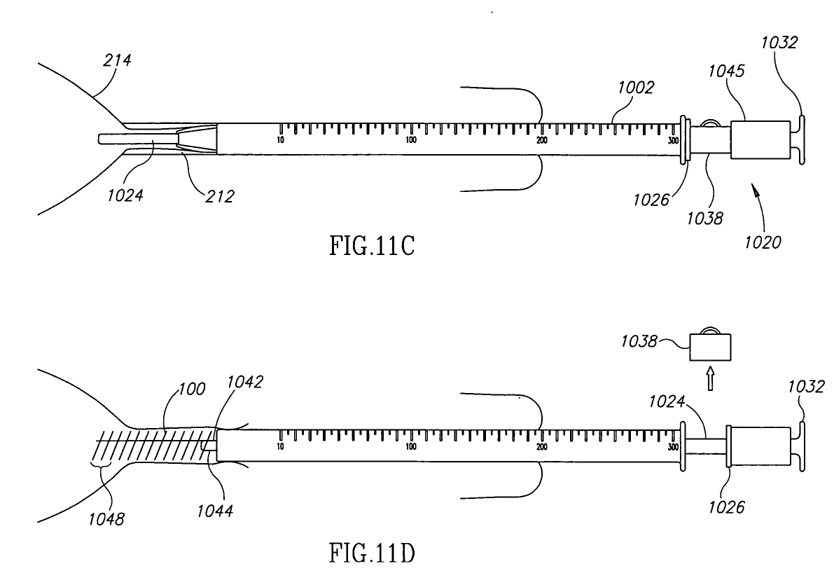

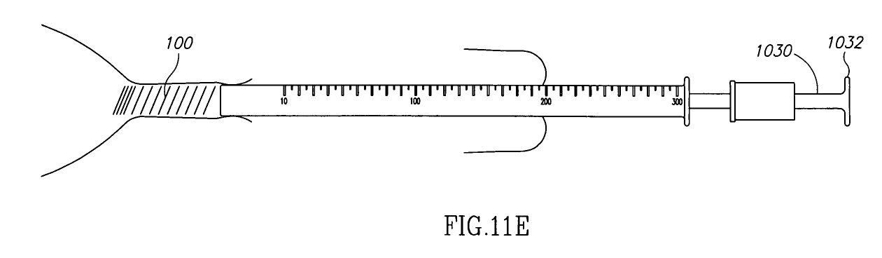

In some embodiments of the invention, as mentioned above, the delivery system may employ an optical fiber used to view the urethra and prevent damage to the sphincter 212 (FIG. 11C). Alternatively or additionally, the delivery may be performed without a balloon on the delivery apparatus. An exemplary embodiment for delivery of implant 100 is described hereinbelow with reference to FIGS. 11A-11E. It is noted that the embodiments of FIGS. 3, 10 and 11A-11E are shown by way of example and additional delivery systems may be used, including combinations of the embodiments of FIGS. 3, 10 and 11A-11E.

Removal Apparatus

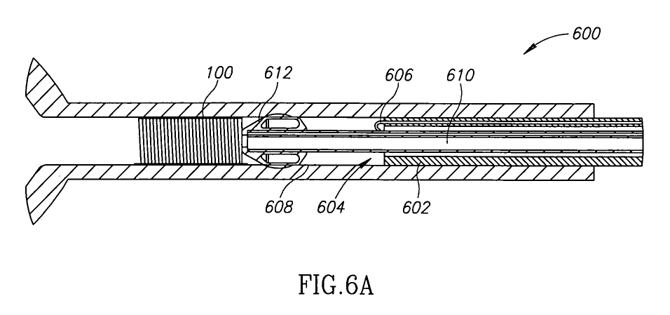

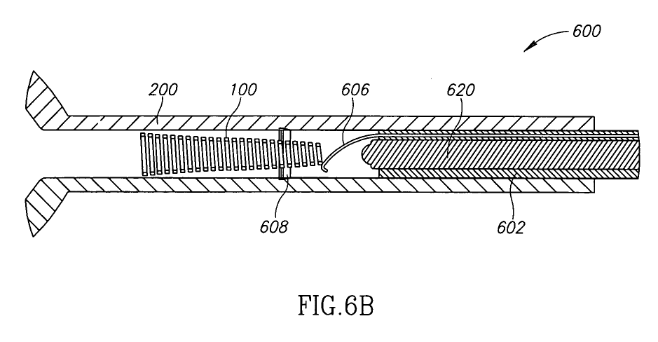

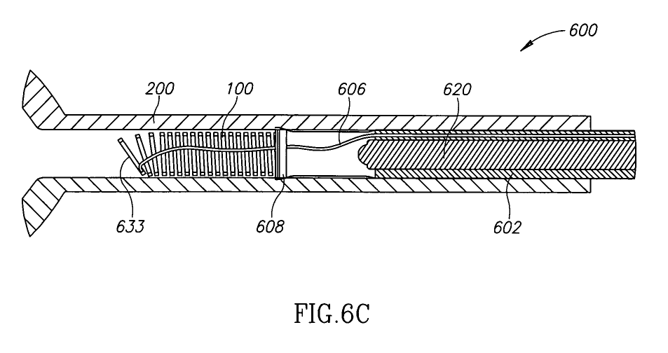

FIGS. 6A and 6B schematically illustrate a process of removing implant 100 from a urethra 200, in accordance with an exemplary embodiment of the invention. As mentioned above, in some embodiments of the invention, when implant 100 completes the dissecting of the obstructing tissue, implant 100 exits the patient's body automatically with the patient's urine. In other embodiments, however, implant 100 is removed using a removal system 600.

Removal system 600 includes an outer tube 608 which surrounds a hook holding body 602 that defines an internal channel 604. Hook holding body 602 carries a hook 606 which is free to move laterally within body 602. During insertion of removal system 600, a balloon catheter 610 is optionally passed through channel 604, with an inflated balloon 612 leading the path into the urethra 200. Once the distal end of body 602 is brought close to implant 100, balloon 612 is deflated and balloon catheter 610 is removed from channel 604.

Referring now to FIG. 6B, in some embodiments of the invention, an optical fiber 620 (or any other viewing apparatus) is inserted into channel 604. Optical fiber 620 may be used, for example, to aid in fitting hook 606 into holes 102 (FIG. 1). Hook 606 is pushed distally toward implant 100, while hook holding body 602 remains in place. Hook 606 is optionally flexible such that it bends radially when it exits body 602. If necessary, the distance between body 602 and implant 100 is adjusted, so as to adjust the point at which hook 606 reaches implant 100 and thus fit hook 606 on to implant 100. The physician then retracts system 600, pulling implant 100 along with hook 606. At first, body 602 is optionally retracted relative to outer tube 608, so as to bring implant 100 into the outer tube. Thereafter, outer tube 608, body 602 and hook 606 are pulled out together from the urethra. Pulling implant 100 into outer tube 608 before removing the implant from the patient reduces the chances of implant 100 getting stuck again on prostate or urethra tissue.

FIG. 6C schematically illustrates a process of removing implant 100 from a urethra 200, in accordance with another exemplary embodiment of the invention. In FIG. 6C, instead of pulling the proximal end of implant 100 as shown in FIG. 6B, hook 606 is passed through implant 100 to the distal turn 633 of the implant. Hook 606 is then used to pull implant 100 through itself into outer tube 608. By pulling implant 100 through itself into outer tube 608, the dragging of implant 100 on the patient's tissue is optionally avoided.

The removal procedure may be performed after the dissecting of the expanding tissue is completed or in emergency cases, before the dissection is completed. Optionally, in such emergency cases, dissection apparatus known in the art (e.g., cryo, ablation, cutting) is used before insertion of removal system 600 to remove the expanded tissue between turns 106, as this tissue anchors implant 100 within prostate 210. In an exemplary embodiment of the invention, implant 100 is removed by stretching it axially, in order to release the tissue caught by the implant.

Other Implant Structures

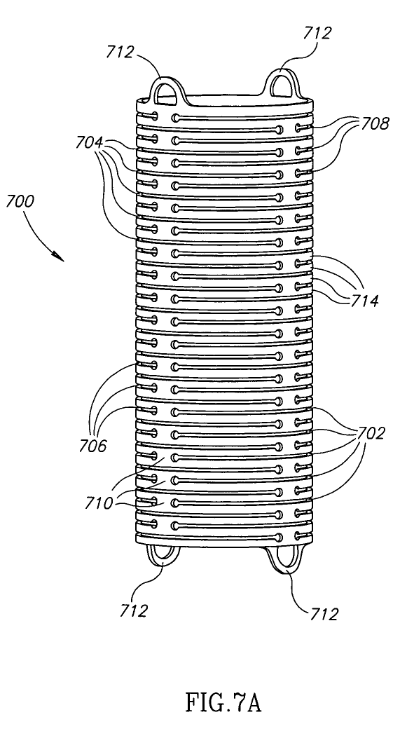

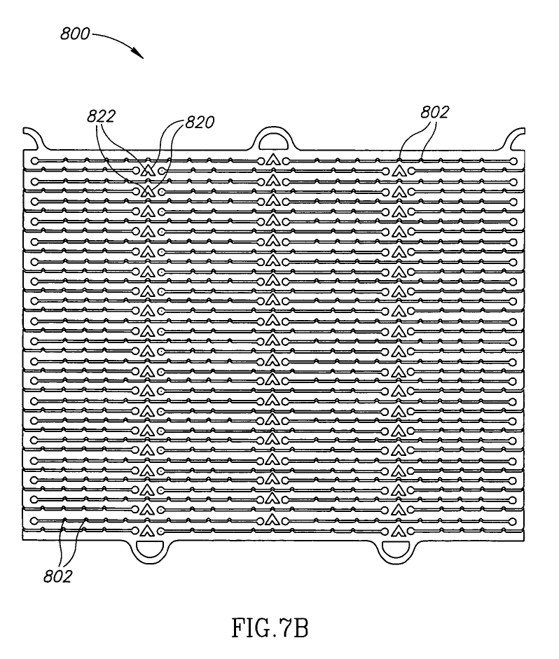

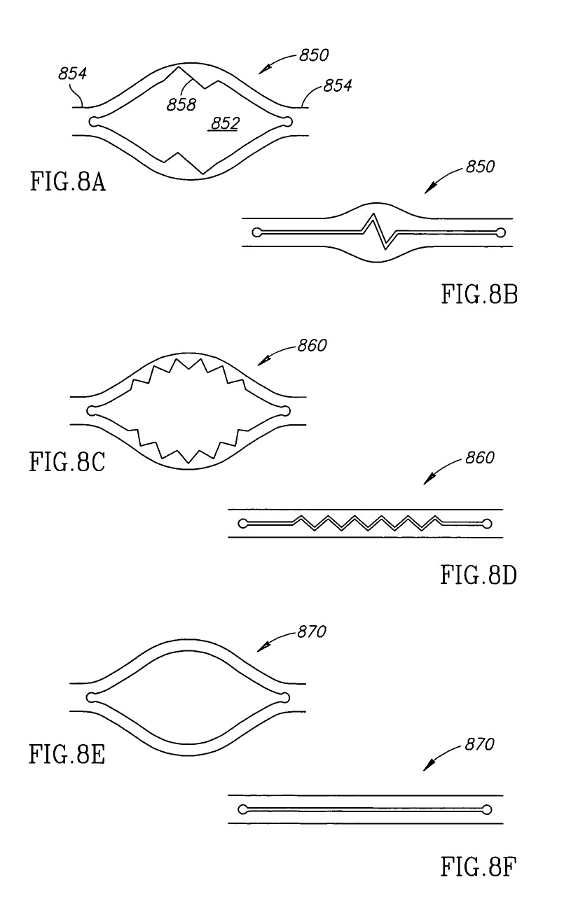

Alternatively to using implant 100, other elastic implant structures may be used. In some embodiments of the invention, an implant is formed of a plurality of springs connected axially by struts. Alternatively or additionally, an implant is formed of a plurality of thin strings connected radially. Optionally, the connection allows a degree of freedom, so that the contraction of each of the springs proceeds at its own pace. Thus, the dissection of soft tissue at one side can proceed at a different rate than dissection of hard tissue on another side. Additional implants are now described, with reference to FIGS. 7A-7B and 8A-8F.

FIG. 7A is a schematic illustration of an elastic prostate implant 700, which may be used instead of implant 100, in accordance with an exemplary embodiment of the invention. Implant 700 comprises an elongate tube having a hollow cylinder shape with a plurality of slits 702, 704, 706 and 708 along its length. The slits divide implant 700 into a plurality of strips 714, which are connected to each other. Stretching implant 100, for example by pulling handles 712 on opposite ends away from each other, opens slits 702, 704, 706 and 708 into gaps which receive obstruction tissue.

Optionally, along the length of implant 100 there are a plurality of slits 702 and opposite side slits 706, at the same axial position along the length of implant 700. Thus, only a small strip 710 of implant 700 and another strip on an opposite side (not shown) connects the portions of implant 700 divided by the slits 702 and 706. Between each two slits 702, slits 704 and 708 are optionally formed at substantially 90° relative to slits 702 and 706. Thus, implant 700 opens tissue grasping gaps in all directions. Alternatively, only slits 702 and 706 are defined in implant 700, so that only obstructing tissue in a portion of the circumference is dissected by implant 700. An implant in accordance with this alternative is optionally used when the obstructing tissue protrudes only from s specific direction.

In some embodiments of the invention, implants 100 and 700 have equal opening area for receiving tissue to be dissected, all around the circumference in 360°. Alternatively, an implant has openings around less than the entire circumference, for example around 270°, 180° or 90°. Such implants may be used, for example, when the obstructing tissue does not cover the entire circumference of the prostate and/or when it is desired to perform the dissection process in a more gradual manner. In an exemplary embodiment of the invention, the dissection is performed in a plurality of stages of insertion of implants. In a first stage, an implant with slots over a limited area of the circumference and/or having a short length is used. If the patient does not react negatively (e.g., with fever), a full scale 360° implant is used in a second stage.

Alternatively to configuring the implant to catch tissue in only some directions, at the time of insertion the implant is prevented from capturing tissue in some of the gaps and/or directions. In some embodiments of the invention, cover 322 (FIG. 3A) is formed of a plurality of separately controllable portions. Optionally, in each circumference portion, cover 322 includes a separate cover. According to the desired directions in which the dissection is to be performed, portions of the cover are removed. The implant is then released to engage tissue in the directions in which the cover was removed but not contact tissue in directions in which the cover was not removed. After the implant (100 or 700) is released, the remaining cover portions are removed together with carrier 350. In some embodiments of the invention, cover 322 includes between 4-6 separate covers. Alternatively or additionally, the implant is inserted into the patient along with one or more barriers which separate the implant from tissue which is not to be dissected. Implant 100 is optionally allowed to move freely relative to the barrier, so that it does not distort.

Due to its structure, implant 700 provides substantially even pressure along its length, without complex production adaptations for changing the applied pressure of different portions of implant 700. It is noted, however, that if it is desired axial portions of implant 700 may be reinforced in order to increase the pressure along portions of the length of implant 700. The reinforcement may be performed by using a larger width or thickness in some axial portions and/or by selective annealing of axial portions of implant 700. It is noted that similar methods may be used to control the pressure of implant 100.

Using implant 700, the dissected tissue is cut off in non-contiguous pieces, each slit 702, 704, 706 and 708 being responsible for a separate fragment of obstructing tissue. Implant 100, in contrast, optionally cuts a contiguous obstructing tissue fragment. In some embodiments of the invention, due to the pressure on the obstructing tissue, the tissue cells die and separate into small tissue fragments.

FIG. 7B is a flattened plan view of an implant 800, in accordance with another exemplary embodiment of the invention. Implant 800 is similar to implant 700, but includes sharp tips 802, which add to the pressure applied to the obstruction tissue and/or help engagement of the tissue by the implant. Sharp tips 802 are optionally pointed in the directions in which the pressure is applied. In some embodiments of the invention, all the sharp tips 802 are in the same direction, so as to allow easier removal of the implant, when necessary. Alternatively, an implant may include sharp tips in both directions in which pressure is applied, so as to increase the pressure on the tissue. Further alternatively or additionally, an implant includes tips in directions orthogonal to the applied pressure and/or diagonal to the direction of the pressure, for example, in order to anchor the implant in the tissue and/or in order to increase the dissection effect radially outward, by pressing the obstruction tissue against the prostate. In other embodiments of the invention, tips extending outward radially are not used, so as not to interfere with the axial contraction of implant 800, while the tissue within the gaps is dissected.

Alternatively or additionally to using small sharp tips 802, large sharp tips are used. In FIG. 7B, slits 820 are formed in implant 800. Tips 822 within the slits 820 are optionally bent outward radially, in order to engage urethra tissue. Tips 822 are optionally bent outward to a small extent, for example of 5-10°. Alternatively or additionally, at least some of tips 822 are bent out of implant 800 by a large extent, of 20-30° or even more, e.g., between 40-70°.

In some embodiments of the invention, tips 822 are extended outwardly at the time of production of the implant or, at the latest, at the time of loading the implant on implant carrier 350. Alternatively or additionally, tips 822 are pushed out after the implant is installed in the patient. Optionally, an internal tube inserted with implant carrier 350 is used to push out tips 822.

Tips 822 are optionally all pointed in a same axial direction. Thus, implant 800 can move axially in one direction, so that implant 800 is allowed to contract axially as the tissue is dissected. Alternatively, tips 822 are directed in both axial directions. Further alternatively or additionally, implant 800 includes tips directed perpendicular to the axial axis of implant 800. Further alternatively or additionally, implant 800 includes tips directed in substantially any other direction.

FIGS. 8A and 8B are schematic illustrations of an implant 850 in open and closed configurations, in accordance with an exemplary embodiment of the invention. Implant 850 includes an elastic mouth 852, which opens when ends 854 are moved closer to each other. In some embodiments of the invention, implant 850 is placed in the patient axially between ends 854. Alternatively, implant 850 is placed horizontally or diagonally.

Before employment, ends 854 are pressed closer to each other in order to open mouth 852. Once obstructing tissue enters into mouth 852, ends 854 are released so mouth 852 closes on the tissue as shown in FIG. 8B.

FIGS. 8C and 8D are schematic illustrations of an implant 860 in open and closed configurations, in accordance with another exemplary embodiment of the invention.

FIGS. 8E and 8F are schematic illustrations of an implant 870 in open and closed configurations, in accordance with another exemplary embodiment of the invention.

Implant 860 includes small teeth which enhance the pressure on the obstructing tissue, by separating the captured tissue into different cavities. This is in contrast to the flat configuration of implant 870 and the configuration of implant 850, in which there is a single large dent 858 (FIG. 8A). It is noted that other configurations may be used according to the texture of the obstructing tissue, the desired dissection time and other dissection related attributes.

Non Elastic Dissection Device

Instead of using an elastic implant which automatically compresses with the progression in the dissection of the obstruction tissue, a tool with a manually adjustable pressure level is used, as is now described. Periodically, a physician or the patient change the mechanical state of the tool, until the dissection is completed.

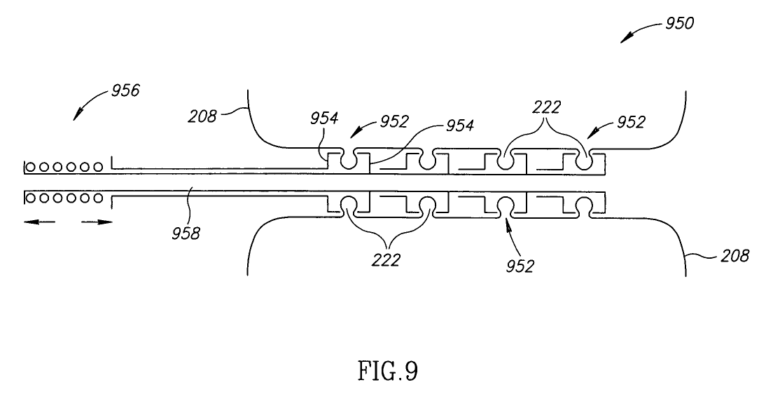

FIG. 9 is a schematic cross-sectional view of a tissue dissection tool 950, in accordance with an exemplary embodiment of the invention. Tool 950 includes a plurality of pressure inducing units 952, formed of opposite members 954. Pressure inducing units 952 catch portions 222 of prostate enlargement 208, so as to dissect these portions. In some embodiments of the invention, a channel 958 passes all along dissection tool 950, allowing urine to pass out through the tool.

A proximal handle 956 is used to control the distance between the members 954 of pressure inducing units 952, and hence the amount of pressure on the prostate portions caught by pressure units 952. In some embodiments of the invention, the patient and/or a physician periodically turn a control on handle 956, so as to increase (or reduce, if necessary) the pressure applied on the dissected tissue. The increase in the applied pressure is optionally performed at predetermined time (e.g., once every 2-3 days). Alternatively or additionally, the increase in the pressure is performed based on feedback on the progression of the dissection, for example from medical acquired images (e.g., ultrasound images). The feedback may also include patient indications on pain or relief. Alternatively or additionally, the feedback is based on the resistance met in turning handle 956.

Alternatively to using a rotating handle 956, a handle which is pulled axially in order to increase the applied force, is used. Alternatively to using channel 958, a thin thread may be used to pull pressure units 952 to a closed position. In some embodiments of the invention, the each pair of members 954 is controlled by a lever (not shown) which closes the gap between the members when the thread is pulled. Alternatively or additionally, the thread is connected to distal members 954 in each unit 952, while a proximal member 954 remains stationary. The thread may include, for example, Kevlar, stainless steel, prolan and/or any other strong material. Alternatively to a single thread, a dissection tool may be controlled by a plurality of threads, which are optionally marked to indicate which units 952 are controlled by each thread. For example, different threads may control units 952 of different cross sectional sectors and/or of different axial areas. The use of threads instead of a massive handle may be more convenient for some patients.

In some embodiments of the invention, a torque limiter, for example using a clutch mechanism, is employed between handle 956 and pressure units 952. The torque limiter limits the force that can be applied to pressure units 952 and prevents inadvertent sudden and/or painful cutting of the tissue.

Optionally, after pressure units 952 are closed entirely, dissection tool 950 is removed from the patient. Alternatively, pressure units 952 are re-opened, and another dissection period is commenced. After pressure units 952 are re-opened, enlargement tissue is allowed to reenter the units for a predetermined period. Thereafter, the pressure units are slowly closed to cause the dissection.

In some embodiments of the invention, all of pressure units 952 are controlled together by handle 956. Alternatively, at least two pressure units 952 are controlled separately. This alternative is more complex, but allows the patient and/or physician more freedom in controlling the dissection. In some embodiments of the invention, the separate control allows controlling different axial portions separately. Alternatively or additionally, the separate control allows control of different radial portions separately.

Handle 956 optionally extends proximally from the patient, to allow simple control by the patient or a physician. Alternatively, the proximal end of handle 956 is within the patient, and a compatible wrench is used to turn the control portion of handle 956. Alternatively or additionally, a pre-programmed automatic controller with a miniature motor or pre-wound spring is embedded within dissection tool 950. The miniature motor is optionally positioned in the bladder and/or in the inner side of the implant. The automatic controller increases the pressure applied by units 952, without need of user intervention.

In some embodiments of the invention, the elasticity of the implant is enhanced and/or replaced by magnets on opposite ends of the implant, which are oriented to attract each other. Alternatively or additionally, one of the ends of the implant is formed of a magnetic material. An external magnet is used to apply force which compresses the implant and applies force on the captured tissue. The size of the magnet may be adjusted in order to control the pressure applied to the tissue caught by the implant.

Alternatively or additionally, implant 100 is configured to contract when a low electrical current is applied to the implant. Implant 100 may include a current source implanted in the patient or an external current source may be used.

In some embodiments of the invention, the urine flow of the patient and/or the chemical form of the urine is used to provide energy used in applying pressure to the tissue by the implant. In an exemplary embodiment of the invention, the implant comprises a material which slowly deforms as it absorbs fluids.

Implant 100 is formed, in some embodiments of the invention, from one or more materials which change with heat and/or under other external conditions, such as electrical current. Applied heat optionally causes the implant to contract and apply pressure to the tissue.

In some embodiments of the invention, implant 100 is formed alternately from two different materials one of which contracts in heat and the other expands in heat. Applying a required heat pattern can cause the implant to exit the patient without requiring an invasive procedure.

Optionally in embodiments which use external force (e.g., magnet, heat), the external force is applied over long periods of time in order to affect the dissection of tissue. Alternatively, the implant includes a ratchet mechanism which prevents the implant from releasing the pressure level it reached. Each time it is required to further compress the implant, the external pressure is applied with a sufficient time and/or power in order to reach the next ratchet level. Further alternatively or additionally, the external force is used in addition to the elasticity of the implant, for example in order to overcome tough tissue and/or in order to enhance the dissection in a beginning and/or ending stage.

Other Insertion Methods

The method of insertion of an implant, shown in FIGS. 3A and 3B was brought as an example and other methods may also be used.

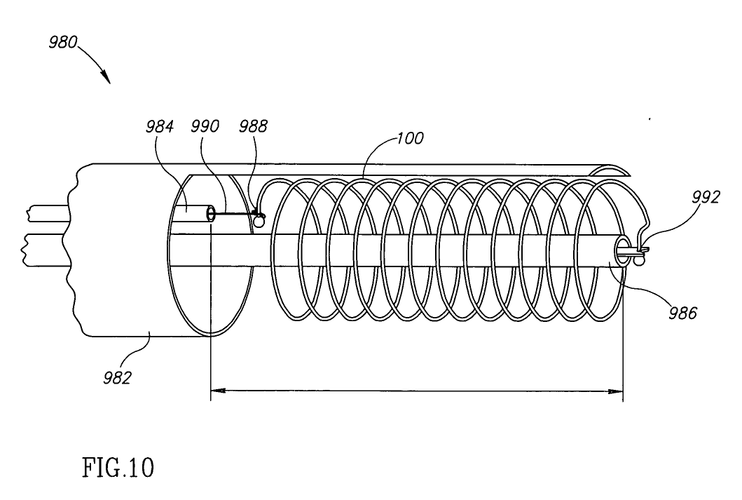

FIG. 10 is a schematic illustration of a system 980 for inserting an implant 100, in accordance with an exemplary embodiment of the invention. System 980 includes a base 982 from which a proximal bar 984 and a distal bar 986 protrude. At their distal ends, bars 984 and 986 are connected to ends of implant 100, optionally with a fast release mechanism. In some embodiments of the invention, the fast release mechanism of proximal bar 984 includes a fast release knot 988. A string 990 to be pulled in order to release knot 988 optionally passes through bar 984. In some embodiments of the invention, distal bar 986 includes a snap mechanism 992. Optionally, releasing snap mechanism 992 requires applying at least a minimal pull force to distal bar 986 relative to implant 100, which is achievable only when implant 100 properly grasps the prostate tissue. Thus, implant 100 cannot be released prematurely.

Alternatively to using different release mechanisms on distal bar 986 and on proximal bar 984, the same mechanism may be used for both bars 984 and 986.

As mentioned above, in some embodiments of the invention, implant 100 is in an axially stretched state when inserted into the patient. Alternatively or additionally to being stretched axially, one end of implant 100 is wound while the other end is held stationary, in a manner which decreases the cross-section radius of implant 100. Referring back to FIGS. 3C and 3D, before an insertion procedure, implant 100 is mounted on implant delivery system 380. In addition to, or instead of, being axially stretched, handle 388 is rotated, while notch 386 grasps a distal end of implant 100, and a proximal end of implant 100 remains stationary or is wound in an opposite direction. After implant 100 is wound sufficiently, its internal radius decreases, such that its insertion into the patient is easier. After implant 100 is inserted into place, handle 388 is rotated in an opposite direction, until the winding of implant 100 is reversed. The reversal of the winding causes he radius of the implant to increase until tissue is caught between the turns of implant 100. Then, the axial stretching of implant 100 by delivery system 380 is released and system 380 is removed from the patient.

Alternatively to releasing the winding of implant 100 by turning handle 380 in an opposite direction, implant 100 may be released abruptly, to expand in an uncontrolled manner.

Referring in detail to the winding of implant, in some embodiments of the invention, the winding reduces the radius of the implant by at least 20%, 40% or even 60%. In an exemplary embodiment of the invention, the winding reduces the radius of the implant from 8 French to less than 5 French or even less than 3 French.

Alternatively to winding implant 100 from its distal end, the winding may be performed from its proximal end, while, for example, notch 386 holds the distal end of the implant.

Safe Insertion

FIGS. 11A-11E illustrate a system and method for accurately accessing prostate tissue, for example for inserting an implant for reshaping prostate tissue, in accordance with an exemplary embodiment of the invention.

FIG. 11A is a schematic illustration of an insertion unit 1000, in accordance with an exemplary embodiment of the invention. Insertion unit 1000 comprises an endoscopic inner tube 104 which is movable within an outer tube 1002. Inner tube 1004 includes a viewing fiber 1006 or any other viewing apparatus, such as an endoscopic camera. At its distal end, inner tube 1004 optionally has a conic shape, to aid in insertion of insertion unit 1000 into the urethra. Outer tube 1002 optionally has a diameter slightly larger (e.g., less than 10% larger) than the diameter of inner tube 1004, allowing relative motion of the tubes relative to each other. In an exemplary embodiment of the invention, inner tube 1004 has a diameter of about 6.8 mm and outer tube 1002 has a diameter of about 7.2 mm. Other diameters may be used according to the size of the patient's urethra.

In some embodiments of the invention, outer tube 1002 includes length markings which allow easy determination of the extent to which insertion unit 1000 is in the urethra. Alternatively, any other method may be used to determine the extent of insertion of insertion unit 1000 into the urethra. For example, tube 1002 may have a position sensor (e.g., a magnet or coil) on its distal end. According to readings of the position sensor, the extent of insertion of tube 1002 is determined.

FIG. 11B is a schematic illustration of insertion unit 1000 within the urethra, in accordance with an exemplary embodiment of the invention. In a first stage, shown in FIG. 11B, optical fiber 1006 is brought to the proximity of sphincter 212. The extent of penetration of insertion unit 1000 is registered when sphincter 212 is viewed in the proximity of the distal end of fiber 1006. In a second stage, outer tube 1002 is pushed to the end of the prostate where prostate meets the bladder 214. Optionally, viewing fiber 1006 is used to verify that outer tube 1002 reached the meeting point of the prostate and bladder 214. The extent of penetration to the distal end of the prostate is then registered. The difference between the extents of penetration at the entrance to the bladder 214 and at sphincter 212 is the length of the prostate. In some embodiments of the invention, according to the determined length, a suitable implant is selected.

Inner tube 1004 is then removed from within outer tube 1002, while outer tube 1002 remains within the urethra.

As shown in FIG. 11C, an implant carrier 1020 is inserted into outer tube 1002 and outer tube 1002 is retracted such that the distal end of outer tube 1002 is at or slightly beyond sphincter 212. The retraction is optionally performed based on the distance registration when viewing fiber 1006 identifies sphincter 212, for example using the markings on outer tube 1002.

In some embodiments of the invention, implant carrier 1020 is first inserted into outer tube 1002 and thereafter outer tube 1002 is retracted. Alternatively, implant carrier 1020 is inserted into outer tube 1002 only after the tube is retracted back to sphincter 212. Further alternatively, outer tube 1002 is not pushed ahead to the entrance to bladder 214, at all.

Implant carrier 1020 optionally includes a protective cover 1024, which isolates the implant from the urethra before it is deployed. Protective cover 1024 optionally has a handle 1026, which may be pulled back in order to expose implant 100, as shown in FIG. 11D. An inner implant holder 1030 (FIG. 11E) of which only a handle 1032 is shown in FIG. 11C, actually carries the implant 100. A handle 1045 allows a physician to hold implant carrier 1020. A locking unit 1038 keeps handle 1026 of protective cover 1024 in place. Removal of locking unit 1038 allows retraction of handle 1026 and exposure of implant 100, as shown in FIG. 11D. In some embodiments of the invention, handle 1026 is spring mounted, such that upon removal of locking unit 1038, implant 100 is automatically exposed.

As shown in FIG. 11D, implant 100 is optionally longer than the prostate enlargement area of most patients, such that a portion of implant 100 is within bladder 214. Once implant 100 is in place and exposed, handle 1032 is retracted, as shown in FIG. 11E, so as to disconnect ties between implant carrier 1020 and implant 100. Optionally, implant 100 is connected to implant holder 1030 through strings 1042 and 1044. Strings 1042 and 1044 optionally have weak points which tear when handle 1032 is retracted. The release of implant 100 optionally causes the implant to anchor in prostate tissue. A portion 1048 (FIG. 11D) of implant 100, located within bladder 214, is not anchored in tissue and therefore contracts axially (as shown schematically in FIG. 11E) upon release of implant 100 from implant holder 1030. The remaining portion of implant 100 anchors in the prostate tissue, so that it does not contract abruptly but rather slowly performs the reshaping.

In some embodiments of the invention, handle 1032 cannot be retracted in order to release implant 100, before handle 1026 was retracted to expose implant 100, so that prostate tissue enters gaps of the implant, anchoring the implant.

After implant 100 is properly positioned, outer tube 1002 and implant carrier 1020 are removed from the urethra.

Alternatively or additionally to recording the extent of penetration of outer tube 1002 into the patient, once the sphincter 212 is identified, outer tube 1002 is slightly advanced and is then anchored in place so that it does not move until after implant 100 is in place.

The method described above with reference to FIGS. 11A-11E may be used also for other prostate treatment methods, such as mechanical cutting of enlargement tissue, cryo-treatment, RF ablation and/or other ablation methods. After positioning outer tube 1002, the cutting and/or treatment tool to be used is passed through outer tube 1002 to the treatment location. Thus, the distal end of outer tube 1002 protects sphincter 112 from the tissue treatment apparatus used.

Although the above description relates to use in the urethra, similar devices can be used in other body channels which may be obstructed, such as in a channel between the kidney and the bladder or between the cholecyst and the liver. Similar devices may optionally be used in the esophagus and/or in the intestine. Furthermore, similar devices to those described above may be used to remove polyps in many body organs. The implant used is adapted to the body portion in which it is employed in the materials used, the size, shape and/or other attributes.

In some embodiments of the invention, system 300 and/or other insertion and/or removal tools are flexible tools which conform to the shape of the body channel into which it is inserted. Alternatively, system 300 and/or other insertion and/or removal tools are rigid, allowing easier insertion of the tools.

It will be appreciated that the above described methods may be varied in many ways. It should also be appreciated that the above described description of methods and apparatus are to be interpreted as including apparatus for carrying out the methods and methods of using the apparatus.

The present invention has been described using non-limiting detailed descriptions of embodiments thereof that are provided by way of example and are not intended to limit the scope of the invention. For example, the removal of the stent may be performed by an endoscopic tweezers, rather than by the apparatus of FIGS. 6A and 6B. It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art.

It is noted that some of the above described embodiments may describe the best mode contemplated by the inventors and therefore may include structure, acts or details of structures and acts that may not be essential to the invention and which are described as examples. Structure and acts described herein are replaceable by equivalents which perform the same function, even if the structure or acts are different, as known in the art. Therefore, the scope of the invention is limited only by the elements and limitations as used in the claims. When used in the following claims, the terms “comprise”, “include”, “have” and their conjugates mean “including but not limited to”.

1. A method of dissecting urethra obstructing tissue, comprising:
capturing urethra obstructing tissue between portions of an implant; and
applying pressure on the tissue caught between the portions of the implant, for longer than 1 hour, until the tissue dies or falls off.

2. A method according to claim 1, wherein the implant applies different pressure levels along a length corresponding to an axis of the urethra.

3. A method according to claim 1, wherein the implant applies substantially equal pressure along its length.

4. A method according to claim 1, wherein the implant comprises an elastic device.

5. A method according to claim 1, wherein applying the pressure comprises applying pressure for at least a day.

6. A method according to claim 1, wherein applying the pressure comprises applying pressure for at least a week.

7. A method according to claim 1, wherein the implant comprises sharp tips pointed outward from the implant.

8. A method according to claim 7, wherein the implant comprises tips pointed in directions in which the implant is adapted to capture tissue.

9. A method according to claim 1, wherein applying the pressure comprises applying pressure parallel to the axis of the urethra.

10. A method according to claim 1, wherein capturing tissue comprises inserting the implant into the urethra in a stretched state and releasing the implant such that it contracts and grasps tissue while contracting.

11. A method according to claim 1, wherein applying the pressure comprises applying by a non-elastic implant.

12. A method according to claim 1, wherein capturing tissue by an implant comprises inserting to the urethra an implant coated by a bio-active material.

13. A method according to claim 12, wherein the bio-active material comprises a tissue dissection drug.

14. A method according to claim 12, wherein the drug comprises a counter inflammation drug.

15. A method according to claim 1, wherein the implant comprises a coil.

16. A method according to claim 15, wherein capturing tissue comprises inserting the coil into the urethra in a winded state and rewinding the coil within the urethra.

17. A method according to claim 15, wherein the coil is formed of a wire having different thicknesses or cross-section shapes along its length.

18. A method according to claim 15, wherein the coil has different length gaps between different pairs of turns of the coil.

19. A method according to claim 15, wherein the coil is pulled inside out in its production process.

20. A method according to claim 1, wherein the implant radially contracts when stretched axially.

21. A tissue dissecting implant kit, comprising:
an implant comprising a plurality of rings coupled to each other elastically, such that an elastic pressure is applied on tissue caught between adjacent rings; and
a sterile package encompassing the implant,
wherein the implant has different distances between adjacent rings along its length or has different material thicknesses or cross-section shapes along its length.

22. An implant kit according to claim 21, wherein the implant comprises a coil.

23. An implant kit according to claim 21, wherein the implant has different distances between adjacent rings along its length.

24. An implant kit according to claim 21, wherein when the implant is in a stretched state resulting from pulling the implant from opposite ends, substantially the same pressure is applied between each pair of adjacent rings.

25. An implant kit according to claim 21, wherein the implant contracts radially when it contracts axially at a first axial stretching extent and expands radially when it contracts axially at a second axial stretching extent.

26. A tissue dissecting implant kit, comprising:
an elongate tube, sized and shaped to fit into a urethra, having a plurality of slits in a circumference of the tube, such that when the tube is in a stretched state along its length it applies a contraction force on tissue within the slits; and
a sterile package in which the tube is packaged.

27. An implant kit according to claim 26, wherein the tube has a substantially cylinder shape in its rest state.

28. A tissue dissecting implant kit, comprising:
an implant comprising a plurality of rings coupled to each other elastically, such that an elastic pressure is applied on tissue caught between adjacent rings; and
a sterile package encompassing the implant,
wherein at least some of the ring surfaces facing each other have non-smooth surfaces.

29. An implant kit according to claim 28, wherein the non-smooth surfaces comprise rough surfaces having a feel similar to sand paper.

30. An implant kit according to claim 28, wherein the non-smooth surfaces comprise small protrusions.

31. An implant kit according to claim 28, wherein the rings are substantially polygonal.

32. An implant kit according to claim 28, wherein the implant comprises a coil.

33. An implant kit according to claim 28, wherein the implant comprises a cylinder with slits.

34. A method of treating the urethra, comprising:
inserting to the urethra an outer tube;
inserting within the outer tube a viewing apparatus;
setting a position of the outer tube in the urethra, which position protects the sphincter, using the viewing apparatus; and
applying a treatment to the urethra while the sphincter is protected by the outer tube.

35. A method according to claim 34, wherein applying the treatment comprises removing the viewing apparatus from the outer tube and inserting a treatment probe through the outer tube.

36. A method according to claim 34, wherein applying the treatment comprises applying RF ablation.

37. A method according to claim 34, wherein applying the treatment comprises cutting tissue with a knife.

38. A method according to claim 34, wherein applying the treatment comprises implanting a tissue dissection implant.

39. A method according to claim 34, wherein the viewing apparatus comprises an optic fiber.

40. An implant delivery system for inserting an implant to the urethra, comprising:
a probe adapted to be inserted to a urethra;
an implant holding unit adapted to hold an axially elastic implant in a stretched state on the probe; and
a release unit adapted to release the implant from the holding unit.

41. An implant delivery system according to claim 40, wherein the implant holding unit is adapted to hold the implant in an axially stretched state.

42. An implant delivery system according to claim 40, wherein the implant holding unit is adapted to hold the implant in a winded state.

43. An implant delivery system according to claim 42, wherein the release unit is adapted to rewind the implant.

44. An implant delivery system according to claim 40, comprising a cover which separates the implant from the urethra until the implant is to be released.

45. An implant delivery system according to claim 40, wherein the implant holding unit comprises strings that are torn by the release unit.

46. A tissue dissecting implant, comprising:
an axially elastic implant adapted for insertion into the urethra; and
a bioactive material coupled to at least a portion of the implant.

47. An implant according to claim 46, wherein the bioactive material comprises a tissue dissection drug.

48. An implant according to claim 46, wherein the bioactive material comprises a counter inflammation drug.

49. An implant according to claim 46, wherein the bioactive material comprises a counter infection drug.

50. An implant according to claim 46, wherein the bioactive material coats at least a portion of an outer surface of the implant.

51. An implant according to claim 46, wherein the bioactive material coats at least some of the member surfaces facing another member.

52. An implant according to claim 46, wherein the bioactive material coats at least a portion of the implant.

53. An implant according to claim 46, wherein the bioactive material is embedded in at least a portion of the implant.

54. An implant according to claim 46, wherein the implant comprises a plurality of members adapted to apply a dissecting force to tissue caught between the members

55. A method of implanting an implant into the urethra, comprising:
mounting the implant in a stretched state onto a probe suitable for insertion into the urethra;
inserting the probe into the urethra, with the implant in the axially stretched state; and
releasing the implant from the probe within the urethra, such that the implant axially condenses.

56. A method according to claim 55, wherein mounting the implant in a stretched state comprises mounting in an axially stretched state.

57. A method according to claim 55, wherein the implant expands radially when it condenses axially.

58. A method according to claim 55, wherein the implant is released from the minimally invasive tool after tissue enters the implant in a manner which dampens the axial contraction of the implant.

59. A method according to claim 55, wherein mounting the implant in a stretched state comprises mounting in a wound state.
